(19) United States
(12) Patent Application Publication
(10) Pub. No.: US 2008/0193524 A1
(43) Pub. Date: Aug. 14, 2008

(54) Extended Release Venlafaxine Compositions

(75) Inventors: Ravinder Kodipyaka, Hyderabad (IN); Subhash Pandurang Gore, Hyderabad (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LTD., Hyderabad, AP (IN); DR. REDDY'S LABORATORIES, INC., Bridgewater, NJ (US)

(21) Appl. No.: 11/997,044

(22) PCT Filed: Jul. 28, 2006
(86) PCT No.: PCT/US06/29343

§ 371 Date: Jan. 28, 2008

Related U.S. Application Data
(60) Provisional application No. 60/786,827, filed on Mar. 28, 2006.

(30) Foreign Application Priority Data
Jul. 28, 2005 (IN) 1022/CHE/2005

(51) Int. Cl. A61K 9/48; A61K 9/30; A61K 31/135

(52) U.S. Cl. 424/463; 424/474; 514/646

(57) ABSTRACT

The invention relates to extended release pharmaceutical compositions of venlafaxine or pharmaceutically acceptable salts. More particularly this invention relates to a pharmaceutical composition having a core comprising venlafaxine and a coating thereupon. The coating utilizes at least one water insoluble polymer and at least one polymer having pH dependent solubility to delay and modulate the release of the drug in an extended manner.

### INTRODUCTION TO THE INVENTION

The present invention relates to extended release pharmaceutical compositions of venlafaxine or pharmaceutically acceptable salts, polymorphs, solvates, single isomers, enantiomers or mixtures thereof and processes for preparing the same.

Venlafaxine is a serotonin-norepinephrine reuptake inhibitor (SNRI) antidepressant. It is chemically named (R,S)-1-[2-(dimethylamino)-1-(4-methoxyphenyl)ethyl]cyclohexanol, or (±)-1-[α-(dimethylamino) methyl]-p-methoxybenzyl]cyclohexanol. It is commercially available as the hydrochloride salt in immediate release tablets under the brand name of EFFEXOR™ and in extended release capsules for once-a-day oral administration under the brand name EFFEXOR™ XR. The extended release capsules contain venlafaxine hydrochloride equivalent to 37.5 mg, 75 mg or 150 mg venlafaxine. The products are manufactured by Wyeth Pharmaceuticals Inc. Venlafaxine hydrochloride has the following structural Formula I.

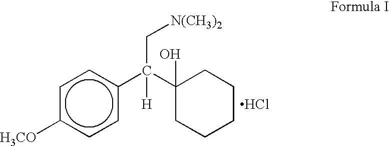

Extended release drug delivery systems are useful in delivering active pharmaceutical ingredients that have one or more of a narrow therapeutic range, short biological half-life and high toxicity. These systems allow dosage delivery by reducing the number of administrations and provide the desired therapeutic effect throughout the day.

U.S. Pat. Nos. 6,274,171, 6,403,120 and 6,419,958 disclose an extended release formulation of venlafaxine hydrochloride for once-daily delivery.

U.S. Patent Application Publication No. 2005/0244498 describes an enhanced absorption delayed controlled release venlafaxine composition for once daily oral administration.

U.S. Patent Application Publication Nos. 2005/0226923 and 2005/0169985 disclose venlafaxine micro tablets and mini tablets, respectively, to be filled into hard gelatin capsules.

U.S. Patent Application Publication Nos. 2005/0042290 and 2003/0091634 disclose controlled release and delayed release venlafaxine compositions, respectively.

U.S. Pat. No. 6,703,044 and U.S. Patent Application Publication No. 2006/0057204 describe delayed burst release formulations of venlafaxine.

Major problems associated with the existing immediate release and extended release formulations of venlafaxine are high incidences of nausea and emesis.

To address this and other such problems, the present invention proposes use of at least one pH dependent polymer, in conjunction with at least one water insoluble polymer, to provide extended venlafaxine release from the pharmaceutical compositions.

### SUMMARY OF THE INVENTION

The present invention relates to extended release pharmaceutical compositions of venlafaxine or pharmaceutically acceptable salts, polymorphs, solvates, single isomers, enantiomers or mixtures thereof and processes for preparing the same.

More particularly this invention relates to a pharmaceutical composition having a core and a coat. The coat utilizes at least one water insoluble polymer and at least one polymer having pH dependent solubility. The polymer having pH dependent solubility activates once it reaches in the stomach and controls the drug release thereafter. The combination of these two polymers provides extended release of venlafaxine, thereby reducing the incidence of undesirable side effects such as nausea and vomiting.

Further, the present invention utilizes a blend of water-insoluble polymer and pH dependent polymer in the coat to delay and modulate the release of the drug in an extended manner for a prolonged or sustained period of time.

In an embodiment, the invention includes a pharmaceutical dosage form comprising a core containing venlafaxine and having a coating comprising a combination of a water-insoluble polymer and a pH-dependent polymer disposed on the core.

In another embodiment, the invention includes a pharmaceutical dosage form comprising multiple cores comprising venlafaxine, the cores having a coating comprising a combination of:

a water-insoluble polymer comprising a cellulose derivative, a polymethacrylic acid-based polymer or copolymer, zein, or an aliphatic polyester; and

a polymer having pH-dependent solubility, comprising a basic butylated methacrylate copolymer, hydroxypropyl methylcellulose phthalate, or cellulose acetate phthalate.

### DETAILED DESCRIPTION OF THE INVENTION

The present invention relates to extended release pharmaceutical compositions of venlafaxine or pharmaceutically acceptable salts, polymorphs, solvates, single isomers, enantiomers or mixtures thereof and processes for preparing the same.

The present invention addresses the problems associated with the use of extended release pharmaceutical compositions of venlafaxine, utilizing a combination of water-insoluble and pH-dependent polymers in the coating to extend the release of the drug, thereby reducing the incidence of undesirable side effects such as nausea and vomiting.

Surprisingly, it was found that the extended release composition of the present invention provides a similar in-vitro drug release profile as compared to marketed EFFEXOR™ XR capsules. However an in-vivo study in healthy human volunteers showed that the time required to reach peak venlafaxine plasma concentrations (Tₘₐₓ) was delayed by 4 to 5 hours, as compared to the reference product EFFEXOR™ XR capsules.

More interestingly, despite showing delayed Tₘₐₓ in an in-vivo study in healthy human volunteers, the extended release composition of the present invention was “bioequivalent” under fed conditions with the marketed EFFEXOR™ XR capsules.

The term “bioequivalent” used herein refers to the ‘sameness’ of the product of present invention and the Reference Listed Drug, according to the established criteria of US Food and Drug Administration and the limits of acceptance thereof.

In one of the embodiments, the composition of the present invention provides Tₘₐₓ in the range between about 8 hours and about 12 hours, or about 10 hours and about 11 hours. This delay in Tₘₐₓ helps in reducing the incidence of nausea and vomiting, which is otherwise detrimental to patient compliance.

An aspect of the present invention provides for extended release compositions comprising a core and one or more coatings applied onto the core.

Achieving an extended release from a core formulation depends on the integrity of core and the type of release controlling coat on the core.

In one embodiment, the compositions comprise a large number of cores comprising active pharmaceutical substance, together with at least one of diluents, binders and other excipients that are commonly used in solid dosage form preparations, such as preservatives, solubilizers, lubricants, and glidants. The said cores are then coated using a blend of a water-insoluble polymer and a pH-dependent polymer.

Examples of cores that are suitable for the present invention include granules, compacted granules, and compressed powders and/or granules. In an embodiment, the core of the present invention comprises mini tablets comprising venlafaxine and at least one water-insoluble polymer, and such core is further coated with extended release polymers.

Extended release coatings utilize a combination of water-insoluble and pH-dependent polymers, along with plasticizers, in the coating to extend the release of the drug.

Useful water-insoluble polymers of various grades used in the coat are exemplified by, but are not limited to: cellulose derivatives such as ethyl cellulose, cellulose acetates and their derivatives, cellulose acylate, cellulose diacylate, cellulose triacylate, cellulose acetate, cellulose diacetate, cellulose triacetate, mono-, di- and tri-cellulose alkanylates, mono-, di-, and tri-cellulose arylates, and mono-, di- and tri-cellulose alkenylates; polymethacrylic acid-based polymers and copolymers such as those sold under the trade name EUDRAGIT™ (RL and RS, NE-30D); zein; and aliphatic polyesters. Other classes of polymers, copolymers of these polymers or their mixtures in various ratios and proportions as required are within the scope of this invention without limitation. Combinations of any two or more of these polymers, and other polymers having the required water-insoluble properties, are within the scope of the invention.

Useful polymers having pH-dependent solubility (soluble in gastric fluid below pH 5) include basic butylated methacrylate copolymers, hydroxypropyl methylcellulose phthalate, cellulose acetate phthalate, and the like. Combinations of any two or more of these polymers, and other polymers having the required pH-dependent solubility properties, are within the scope of the invention.

Of course, any other polymers, or combinations thereof, which demonstrate such characteristics like water-insolubility and/or pH-dependent solubility and are useful for the composition to modulate the release of the venlafaxine, are also acceptable in the working of this invention.

In one embodiment, water-insoluble polymers or hydrophobic polymers such as ethyl cellulose have been found to be useful for the composition, in combination with pH-dependent polymers such as butylated methacrylate copolymers in the coating to modulate the release of the drug in a desired manner for an extended period of time. Examples of useful polymers are EUDRAGIT™ E and EUDRAGIT™ E PO, aminoalkyl methacrylate copolymers having tertiary ammonium functional groups, chemically described as poly(butyl methacrylate), (2-dimethylaminoethyl)methacrylate, methyl methacrylate 1:2:1 and manufactured by Röhm GmbH & Co, KG of Darmstadt, Germany.

According to the present invention, the weight ratio of a water-insoluble polymer to a pH-dependent polymer for the coating composition ranges from 1:15 to 15:1, or from 1:13 to 13:1, or from 1:11 to 11:1, or from 1:8 to 8:1. Other useful weight ratios of water-insoluble polymer to pH-dependent polymer range from 4:1 to about 12:1, or about 5:1 to about 10:1; such ratios have been found appropriate for venlafaxine hydrochloride compositions having ethyl cellulose as the water-insoluble polymer and EUDRAGIT E or EUDRAGIT E PO as pH-dependent polymers. The ratios depend to a degree on the characteristics of the polymers that are chosen, and an appropriate ratio for a desired drug release profile can easily be determined by experimentation, such as following the procedures in the examples below.

The cores of active substance coated with rate controlling polymers can be formulated as tablets or beads that are filled into hard gelatin capsules, sachets and the like to obtain the desired in vivo release profiles after administration.

In the context of the present invention, during the preparation of the pharmaceutical compositions into finished dosage form, one or more pharmaceutically acceptable excipients may optionally be used which include but are not limited to: diluents such as microcrystalline cellulose (MCC), silicified MCC (e.g. PROSOLV™ HD 90), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders such as acacia, guar gum, alginic acid, dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethyl cellulose sodium, povidone (various grades of KOLLIDON®, PLASDONE®), starch and the like; chelating agents like edetate disodium; surfactants including anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, sodium lauryl sulfate (SLS) and sodium dodecyl sulfate (SDS); cationic surfactants such as cetylpyridinium chloride monohydrate and hexadecyltrimethylammonium bromide; nonionic surfactants such as N-decanoyl-N-methylglucamine, octyl a-D-glucopyranoside, n-Dodecyl b-D-maltoside (DDM), polyoxyethylene sorbitan esters like polysorbates and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, medium chain triglycerides (e.g., Miglyol™ 812N), fatty alcohols such as cetostearyl alcohol, cetyl alcohol, stearyl alcohol, oleyl alcohol, myristyl alcohol and the like; solvents that may be used in granulation or layering or coating are aqueous such as water, or alcoholic such as ethanol, isopropanolol, or hydro-alcoholic such as a mixture of water with alcohol in any ratio, or organic such as acetone, methylene chloride, dichloromethane and the like.

Pharmaceutical compositions of the present invention may further include any one or more of pharmaceutically acceptable glidants, lubricants, opacifiers, colorants and other commonly used excipients.

In other embodiments, the pharmaceutical compositions of the present invention are filled into capsules (e.g., hard gelatin capsules). Pharmaceutical compositions to be filled into capsules can include pharmaceutically acceptable excipients, for example diluents such as cellulose, microcrystalline cellulose, mannitol, starch, and pregelatinized starch, and/or flow aids such as stearates.

The present invention provides a unit dose of venlafaxine of about 10 to about 200 milligrams per dosage form.

The following examples will further illustrate certain specific aspects and embodiments of the invention in greater detail and are not to be construed as limiting the scope of the invention.

### EXAMPLE 1

### Compositions for Venlafaxine Extended Release Capsules

| | |
|---|---|
| Ingredients | mg/Capsule |

Core

| | |
|---|---|
| Venlafaxine hydrochloride | 170 |
| Microcrystalline cellulose | 112 |
| (AVICEL PH 112)* | |
| Povidone K 90** | 17.7 |
| Ethyl cellulose (7 cps) | 41.8 |
| Sodium stearyl fumarate | 7 |
| Talc | 3.5 |

Coating

| | |
|---|---|
| Ethyl cellulose (7 cps) | 33.6 |
| Medium chain triglycerides | 3.4 |
| (MIGLYOL 812N)# | |
| Basic butylated methacrylate | 5.3 |
| copolymer (EUDRAGIT E) | |

*FMC Biopolymer manufactures AVICEL PH 112.
**ISP manufactures Povidone K 90.
#MIGLYOL 812N is manufactured by BASF.

### Manufacturing Process:

1. Venlafaxine hydrochloride and Avicel PH 112 were sifted through a 25# mesh sieve.

2. Material of step 1 was blended in double cone blender for 10 minutes.

3. Povidone K 90 was dissolved in isopropanol using an overhead stirrer.

4. Blend of step 2 was granulated with binder solution of step 3.

5. Wet granules of step 4 were dried in fluid bed dryer at 50° C. till the loss on drying of granules was between 1-2% w/w.

6. Dried granules of step 5 were passed through a 24# mesh sieve.

7. Granules of step 6 were blended with ethyl cellulose in a double cone blender for 10 minutes.

8. Blend of step 7 was lubricated with talc and sodium stearyl fumarate in a double cone blender for 10 minutes.

9. Blend of step 8 was compressed into mini-tablets using a 2.5 mm punch.

10. Ethyl cellulose was dissolved in isopropanol under stirring.

11. Miglyol 812N was added to the step 10 solution and the solution was stirred for 15 minutes.

12. Eudragit E was dissolved in isopropanol.

13. Solution of step 12 was added to step 11 and stirred for 10 minutes.

14. Mini tablets of step 9 were coated with solution of step 13 in a perforated coating pan.

15. Resulting coated mini-tablets equivalent to 150 mg of venlafaxine were filled into hard gelatin capsules

The capsules were tested using the following in-vitro dissolution conditions, yielding the following results:

Apparatus: USP type 1

Stirring speed: 100 rpm

Dissolution medium: 0.1 N HCl (2 hours), followed by pH 6.8 phosphate buffer

Volume of dissolution medium: 900 ml

| Time (hours) | % Drug Released |
|---|---|
| 2 | 13 |
| 4 | 45 |
| 8 | 68 |
| 12 | 80 |
| 24 | 92 |

An in-vivo pharmacokinetic study of the capsules was carried out with administration in the fed state on healthy volunteers (n−12). The pharmacokinetic parameters are given in the following table:

| Pharmacokinetic | |
|---|---|
| Parameters | Example 1 |
| Tₘₐₓ (hours) | 9 |
| Cₘₐₓ (ng/ml) | 126.2 |
| AUC₍₀₋ₜ₎ (hr · ng/ml) | 1958.5 |
| AUC_{(0-inf)} (hr · ng/ml) | 2017.6 |

### EXAMPLE 2

### Compositions for Venlafaxine Extended Release Capsules

| | |
|---|---|
| Ingredients | mg/Capsule |

Core

| | |
|---|---|
| Venlafaxine hydrochloride | 170 |
| Microcrystalline cellulose | 112 |
| (AVICEL PH 112) | |
| Povidone K 90 | 17.7 |
| Ethyl cellulose (7 cps) | 41.8 |
| Sodium stearyl fumarate | 7 |
| Talc | 3.5 |

Coating

| | |
|---|---|
| Ethyl cellulose (7 cps) | 43.5 |
| Medium chain triglycerides | 4.4 |
| (MIGLYOL 812N) | |
| Basic butylated methacrylate | 8.5 |
| copolymer (EUDRAGIT E) | |

Manufacturing process was the same as that described in Example 1.

### In-Vitro Dissolution Conditions:

Apparatus: USP type 1

Stirring speed: 100 rpm

Dissolution medium: 0.1 N HCl (2 hours), followed by pH 6.8 phosphate buffer

Volume of dissolution medium: 900 ml

| Time (hours) | % Drug Released |
|---|---|
| 2 | 20 |
| 4 | 52 |
| 8 | 66 |
| 12 | 73 |

### EXAMPLE 3

### Compositions for Venlafaxine Extended Release Capsules 150 mg

| | |
|---|---|
| | Batch Quantities |
| Ingredients | (kg) |

Core

| | |
|---|---|
| Venlafaxine hydrochloride | 46.67 |
| Mocrocrystalline cellulose | 30.91 |
| (AVICEL PH112) | |
| Povidone K90 | 4.84 |
| Ethyl cellulose (7 cps) | 11.50 |
| Sodium stearyl fumarate | 1.93 |
| Talc | 0.96 |
| Isopropyl alcohol | 33 |

Coating

| | |
|---|---|
| Ethyl cellulose (7 cps) | 11.01 |
| Medium chain triglycerides | 1.10 |
| (MIGLYOL 812N) | |
| Basic butylated methacrylate | 1.73 |
| copolymer (EUDRAGIT E PO) | |
| Isopropyl alcohol | 217 |

Capsule filling

| | |
|---|---|
| Size ‘0 el’ hard gelatin capsules | Approx. 275,000 |

Manufacturing process was the same as that described in Example 1.

### In Vitro Dissolution Conditions:

Apparatus: USP type 1

Stirring speed: 100 rpm

Dissolution medium: 0.1 N HCl (2 hours), followed by pH 6.8 phosphate buffer

Volume of dissolution medium: 900 ml

% Drug Released

| Time (hours) | Example 3 | Effexor ™ XR 150 mg |
|---|---|---|
| 2 | 10 | 14 |
| 4 | 43 | 44 |
| 8 | 66 | 65 |
| 12 | 79 | 76 |
| 24 | 92 | 89 |

A comparative in-vivo pharmacokinetic study of the Example 3 capsules and the commercial EFFEXOR™ XR capsule product was carried out with administration in a fed state using healthy volunteers (n=36). The pharmacokinetic parameters are given in the following table:

| Pharmacokinetic | | EFFEXOR ™ |
|---|---|---|
| Parameter | Example 3 | XR 150 mg |
| Tₘₐₓ (hours) | 10.4 | 6 |
| Cₘₐₓ (ng/ml) | 142.1 | 156.7 |
| AUC₍₀₋ₜ₎ (hr · ng/ml) | 2561.7 | 2519.9 |
| AUC_{(0-inf)} (hr · ng/ml) | 2675.3 | 2656.2 |

### EXAMPLES 4-6

### Compositions for Venlafaxine Extended Release Capsules 150 mg

| | Batch Quantities (gm) | | |
|---|---|---|---|
| Ingredients | Example 4 | Example 5 | Example 6 |

Core

| | | | |
|---|---|---|---|
| Venlafaxine hydrochloride | 237.6 | 237.6 | 237.6 |
| Microcrystalline cellulose | 205.5 | 205.5 | 205.5 |
| Plasdone S630 | 35 | 35 | 35 |
| Sodium stearyl fumarate | 9.8 | 9.8 | 9.8 |
| Talc | 4.9 | 4.9 | 4.9 |
| Isopropyl alcohol | 119.5 | 119.5 | 119.5 |

Coating

| | | | |
|---|---|---|---|
| Ethyl cellulose (7 cps) | 60.5 | 68.5 | 70.6 |
| Medicum chain tryglycerides | 6 | 6.9 | 7.1 |
| (MIGLYOL 812N) | | | |
| Basic butylated mathacrylate | 7.4 | 13.3 | 11.1 |
| copolymer (Eudragit E PO) | | | |
| Isopropyl alcohol | 1157 | 1389 | 1391 |

Manufacturing process was the same as that described in Example 1.

### In Vitro Dissolution Conditions:

Apparatus: USP type 1

Stirring speed: 100 rpm

Dissolution medium: 0.1 N HCl (2 hours) followed by pH 6.8 phosphate buffer

Volume of dissolution medium: 900 ml

| | % Drug Released | | |
|---|---|---|---|
| Time (hours) | Example 4 | Example 5 | Example 6 |
| 2 | 4 | 73 | 23 |
| 4 | 27 | 83 | 49 |
| 8 | 42 | 96 | 70 |
| 12 | 57 | 98 | 80 |

### EXAMPLES 7-8

### Compositions for Venlafaxine Extended Release Capsules 150 mg

Batch Quantities (gm)

| | | |
|---|---|---|
| Ingredients | Example 7 | Example 8 |

Core

| | | |
|---|---|---|
| Venlafaxine hydrochloride | 339.4 | 339.4 |
| Microcrystalline cellulose | 210 | 224.8 |
| Povidone K 30 | 50 | 35.2 |
| Ethyl cellulose (7 cps) | 83.6 | 83.6 |
| Sodium stearyl fumarate | 14 | 14 |
| Talc | 7 | 7 |
| Isopropyl alcohol | 336 | 336 |

Coating

| | | |
|---|---|---|
| Ethyl cellulose (7 cps) | 70.6 | 70.6 |
| Medium chain tryglycerides | 7.1 | 7.1 |
| (MIGLYOL 812N) | | |
| Basic butylated mathacrylate | 11.1 | 11.1 |
| copolymer (Eudragit E PO) | | |
| Isopropyl alcohol | 1391 | 1391 |

Manufacturing process was the same as that described in Example 1.

1. A pharmaceutical dosage form comprising a core containing venlafaxine and having a coating comprising a combination of a water-insoluble polymer and a pH-dependent polymer disposed on the core.

2. The pharmaceutical dosage form of claim 1, wherein venlafaxine is present in the form of a salt.

3. The pharmaceutical dosage form of claim 1, wherein venlafaxine is present as a hydrochloride salt.

4. The pharmaceutical dosage form of claim 1, wherein a water-insoluble polymer comprises a cellulose derivative, a polymethacrylic acid-based polymer or copolymer, zein, or an aliphatic polyester.

5. The pharmaceutical dosage form of claim 1, wherein a water-insoluble polymer comprises a cellulose derivative.

6. The pharmaceutical dosage form of claim 1, wherein a water-insoluble polymer comprises ethyl cellulose.

7. The pharmaceutical dosage form of claim 1, wherein a pH-dependent polymer comprises a basic butylated methacrylate copolymer, hydroxypropyl methylcellulose phthalate, or cellulose acetate phthalate.

8. The pharmaceutical dosage form of claim 1, wherein a pH-dependent polymer comprises a basic butylated methacrylate copolymer.

9. The pharmaceutical dosage form of claim 1, wherein a coating comprises a plasticizer.

10. The pharmaceutical dosage form of claim 1, wherein a weight ratio of water-insoluble polymer to pH-dependent polymer is from 1:15 to 15:1.

11. The pharmaceutical dosage form of claim 1, wherein a weight ratio of water-insoluble polymer to pH-dependent polymer is from 1:13 to 13:1.

12. The pharmaceutical dosage form of claim 1, wherein a weight ratio of water-insoluble polymer to pH-dependent polymer is from 1:11 to 11:1.

13. The pharmaceutical dosage form of claim 1, wherein a weight ratio of water-insoluble polymer to pH-dependent polymer is from 1:8 to 8:1.

14. The pharmaceutical dosage form of claim 1, wherein a weight ratio of water-insoluble polymer to pH-dependent polymer is from 4:1 to 12:1.

15. The pharmaceutical dosage form of claim 1, wherein a weight ratio of water-insoluble polymer to pH-dependent polymer is from 5:1 to 10:1.

16. The pharmaceutical dosage form of claim 1, wherein a core is a mini-tablet.

17. The pharmaceutical dosage form of claim 1, having multiple coated cores contained in a capsule.

18. A pharmaceutical dosage form comprising multiple cores comprising venlafaxine, the cores having a coating comprising a combination of:
a water-insoluble polymer comprising a cellulose derivative, a polymethacrylic acid-based polymer or copolymer, zein, or an aliphatic polyester; and
a polymer having pH-dependent solubility, comprising a basic butylated methacrylate copolymer, hydroxypropyl methylcellulose phthalate, or cellulose acetate phthalate.

19. The pharmaceutical dosage form of claim 18, wherein venlafaxine is present as a hydrochloride salt.

20. The pharmaceutical dosage form of claim 18, wherein a water-insoluble polymer comprises a cellulose derivative.

21. The pharmaceutical dosage form of claim 18, wherein a water-insoluble polymer comprises ethyl cellulose.

22. The pharmaceutical dosage form of claim 18, wherein a polymer having pH-dependent solubility comprises a basic butylated methacrylate copolymer.

23. The pharmaceutical dosage form of claim 18, wherein venlafaxine is present as a hydrochloride salt, a water-insoluble polymer comprises ethyl cellulose, a pH-dependent polymer comprises a basic butylated methacrylate copolymer, and a weight ratio of water-insoluble polymer to pH-dependent polymer is from 4:1 to 12:1.

24. The pharmaceutical dosage form of claim 18, wherein venlafaxine is present as a hydrochloride salt, a water-insoluble polymer comprises ethyl cellulose, a pH-dependent polymer comprises a basic butylated methacrylate copolymer, and a weight ratio of water-insoluble polymer to pH-dependent polymer is from 5:1 to 10:1.

25. The pharmaceutical dosage form of claim 18, wherein a core is a mini-tablet.

26. The pharmaceutical dosage form of claim 1 providing, following administration to a human, a maximum plasma concentration of venlafaxine between about 8 and about 12 hours.

27. The pharmaceutical dosage form of claim 1 providing, following administration to a human, a maximum plasma concentration of venlafaxine between about 10 and about 11 hours.

28. The pharmaceutical composition of claim 1, wherein a core further contains a water-insoluble polymer.

29. The pharmaceutical composition of claim 18, wherein cores further contain a water-insoluble polymer.

30. The pharmaceutical dosage form of claim 16, wherein the mini-tablet has been formed by compressing a powder, granules, or a mixture thereof.

31. The pharmaceutical dosage form of claim 25, wherein the mini-tablet has been formed by compressing a powder, granules, or a mixture thereof.

32. A pharmaceutical dosage form comprising a core containing a venlafaxine salt and a water-insoluble polymer, the core having a coating disposed thereon comprising a combination of a water-insoluble polymer and a pH-dependent polymer.

33. The pharmaceutical dosage form of claim 32, wherein the core has been formed by compressing a powder, granules, or a mixture thereof.
